(19) United States
(12) Patent Application Publication
(10) Pub. No.: US 2024/0130890 A1
(43) Pub. Date: Apr. 25, 2024

(54) SUSTAINED-RELEASE IMPLANTS FOR LOWERING INTRAOCULAR PRESSURE WITH EXTENDED DURATION OF EFFECT

(71) Applicant: Allergan, Inc., Irvine, CA (US)

(72) Inventors: Michael R. Robinson, Huntington Beach, CA (US); Marina Bejanian, Laguna Niguel, CA (US); Michelle Y. Chen, Irvine, CA (US)

(21) Appl. No.: 18/200,064

(22) Filed: May 21, 2023

Related U.S. Application Data
(63) Continuation of application No. 16/184,834, filed on Nov. 8, 2018, now abandoned.
(60) Provisional application No. 62/683,337, filed on Jun. 11, 2018.
(60) Provisional application No. 62/583,967, filed on Nov. 9, 2017.

(51) Int. Cl. A61F 9/00; A61F 9/007; A61K 9/00; A61K 31/5575; A61K 47/10; A61K 47/34; A61P 27/06

(52) U.S. Cl. CPC A61F 9/0017; A61F 9/00781; A61K 9/0051; A61K 31/5575; A61K 47/10; A61K 47/34; A61P 27/06

(57) ABSTRACT

Methods for treatment of increased intraocular pressure with intracameral intraocular implants are disclosed herein. The controlled and sustained release of bimatoprost to the anterior chamber of the eye may be effective to treat an eye for at least one year or longer for the reduction of TOP.

### CROSS-REFERENCE TO RELATED APPLICATIONS

This application claims the benefit of U.S. Provisional Application No. 62/583,967 filed on Nov. 9, 2018 and U.S. Provisional Application No. 62/683,337 filed on Jun. 11, 2018, the entire content of which are incorporated herein by reference.

### BACKGROUND

### Field

The disclosure of the present application generally relates to drug delivery implants, and more specifically, drug delivery implants used to treat ocular conditions.

### Description of the Related Art

Poor adherence to topical intraocular pressure (IOP)—lowering medication is common in glaucoma, associated with worse outcomes.¹ Because of these limitations, biodegradable sustained-release implant containing bimatoprost, such as those described in U.S. Patent Application Publication No. 2015-0118279, which is herein incorporated by reference in its entirety (including disclosures of formulations, implant dimensions, methods of use, and polymer compositions and properties), has developed to address nonadherence in the glaucoma population. Other bimatoprost-containing intraocular implants have been described in U.S. Pat. No. 7,799,336, which is incorporated by reference in its entirety. While these publications describe implants which deliver drug to a patient's eye over an extended period of time, it is desirable to develop a drug which has an extended duration of clinical effect, for example, an implant that can reduce a patient's intraocular pressure over at least one year.

### SUMMARY

Accordingly, an embodiment provides a method of reducing intraocular pressure (IOP) in an eye of a patient including injecting a single intraocular implant comprising bimatoprost or a salt thereof and a biodegradable polymer into the anterior chamber of a patient in need thereof, where the intraocular implant is effective to reduce the IOP of the patient in need thereof over a period of time between about 12 months and about 24 months. In some embodiments, the intraocular implant is effective to reduce the IOP of the patient in need thereof of a period of time of about 24 months. In some embodiments the intraocular implant includes 6 μg, 10 μg, 15 μg or 20 μg of bimatoprost or a salt thereof. In an embodiment, the intraocular implant includes a biodegradable polymer matrix, polyethylene glycol 3350, and bimatoprost or a salt thereof, wherein the bimatoprost or a salt thereof and polyethylene glycol 3350 are associated with the biodegradable polymer matrix, which includes an ester end poly(D,L-lactide) having an inherent viscosity of 0.25 dl/g to 0.35 dl/g, an acid end poly(D,L-lactide) having an inherent viscosity of 0.16 dl/g to 0.24 dl/g, and an ester end poly(D,L-lactide-co-glycolide) having an inherent viscosity of 0.16 dl/g to 0.24 dl/g and a D,L-lactide to glycolide molar ratio of about 75:25, wherein the bimatoprost or a salt thereof constitutes 18% to 22% of the implant by weight, the ester end poly(D,L-lactide) constitutes 18% to 22% of the implant by weight, the acid end poly(D,L-lactide) constitutes 13.5% to 16.5% of the implant by weight, the ester end poly(D,L-lactide-co-glycolide) constitutes 36% to 44% of the implant by weight, and wherein the polyethylene glycol 3350 constitutes 3.5% to 6.5% of the implant by weight, wherein the inherent viscosity of each of the poly(D,L-lactide) and poly(D,L-lactide-co-glycolide) polymers is determined for a 0.1% solution of the polymer in chloroform at 25° C.

In an embodiment, a method of treating open angle glaucoma or ocular hypertension in a patient includes injecting one or more intraocular implants comprising bimatoprost or a salt thereof and a biodegradable polymer into the anterior chamber of the eye of a patient in need thereof at a frequency of one intraocular implant every four months to one intraocular implant every twelve months over a treatment period including injection of a first intraocular implant and a final intraocular implant. The intraocular implants can be effective to reduce the IOP of the patient in need thereof over a period of time between about 12 months and about 24 months after injection of the final intraocular implant. According to some embodiments, the patient receives one intraocular implant every four months, and the intraocular implant or implants are effective to reduce the TOP of the patient in need thereof over a period of time between about 12 months and about 24 months. In an embodiment, the patient receives between two implants and eight implants over the treatment period. In some embodiments, the patient receives three implants. According to other embodiments, the intraocular implants are effective to reduce the TOP of the patient in need thereof over a period of time of about 12 months. The intraocular implant can include 6 μg, 10 μg, 15 μg of bimatoprost or a salt thereof. In an embodiment, the intraocular implant includes a biodegradable polymer matrix, polyethylene glycol 3350, and bimatoprost or a salt thereof, wherein the bimatoprost or a salt thereof and polyethylene glycol 3350 are associated with the biodegradable polymer matrix, which includes an ester end poly(D,L-lactide) having an inherent viscosity of 0.25 dl/g to 0.35 dl/g, an acid end poly(D,L-lactide) having an inherent viscosity of 0.16 dl/g to 0.24 dl/g, and an ester end poly(D,L-lactide-co-glycolide) having an inherent viscosity of 0.16 dl/g to 0.24 dl/g and a D,L-lactide to glycolide molar ratio of about 75:25, wherein the bimatoprost or a salt thereof constitutes 18% to 22% of the implant by weight, the ester end poly(D,L-lactide) constitutes 18% to 22% of the implant by weight, the acid end poly(D,L-lactide) constitutes 13.5% to 16.5% of the implant by weight, the ester end poly(D,L-lactide-co-glycolide) constitutes 36% to 44% of the implant by weight, and wherein the polyethylene glycol 3350 constitutes 3.5% to 6.5% of the implant by weight, wherein the inherent viscosity of each of the poly(D,L-lactide) and poly(D,L-lactide-co-glycolide) polymers is determined for a 0.1% solution of the polymer in chloroform at 25° C.

According to some embodiments, a method of treating open angle glaucoma or ocular hypertension in a patient includes injecting one or more single intraocular implants that include bimatoprost or a salt thereof and a biodegradable polymer into the anterior chamber of the eye of a patient in need thereof at a frequency of one intraocular implant every four months to one intraocular implant every twelve months over a treatment period including injection of a first intraocular implant and a final intraocular implant. In such embodiments, after injection of the final implant, the patient does not require rescue medication for a period of time between about 12 months and about 24 months. In some embodiments, the patient receives one intraocular implant every four months and the patient does not require rescue medication over a period of time between about 12 months and about 24 months after injection of the final implant. According to some embodiments, the patient receives between two implants and eight implants over the treatment period. The patient can receive three implants. In an embodiment, the patient does not require rescue medication over a period of time of about 12 months. The intraocular implant can include, in some embodiments, 6 μg, 10 μg, or 15 μg of bimatoprost or a salt thereof. In an embodiment, the intraocular implant includes a biodegradable polymer matrix, polyethylene glycol 3350, and bimatoprost or a salt thereof, wherein the bimatoprost or a salt thereof and polyethylene glycol 3350 are associated with the biodegradable polymer matrix, which includes an ester end poly(D,L-lactide) having an inherent viscosity of 0.25 dl/g to 0.35 dl/g, an acid end poly(D,L-lactide) having an inherent viscosity of 0.16 dl/g to 0.24 dl/g, and an ester end poly(D,L-lactide-co-glycolide) having an inherent viscosity of 0.16 dl/g to 0.24 dl/g and a D,L-lactide to glycolide molar ratio of about 75:25, wherein the bimatoprost or a salt thereof constitutes 18% to 22% of the implant by weight, the ester end poly(D,L-lactide) constitutes 18% to 22% of the implant by weight, the acid end poly(D,L-lactide) constitutes 13.5% to 16.5% of the implant by weight, the ester end poly(D,L-lactide-co-glycolide) constitutes 36% to 44% of the implant by weight, and wherein the polyethylene glycol 3350 constitutes 3.5% to 6.5% of the implant by weight, wherein the inherent viscosity of each of the poly(D,L-lactide) and poly(D,L-lactide-co-glycolide) polymers is determined for a 0.1% solution of the polymer in chloroform at 25° C. According to some embodiments of the method, the rescue medication includes eye drops containing a prostaglandin analog or prostamide. In some embodiments, the method is effective to reduce the IOP of the patient in need thereof by about 30% from baseline.

### BRIEF DESCRIPTION OF THE FIGURES

These and other features will now be described with reference to the drawings summarized below. These drawings and the associated description are provided to illustrate one or more embodiments and not to limit the scope of the invention.

FIG. 1 illustrates a clinical study dosing design, according to example embodiments including the study design described in EXAMPLE 2.

FIG. 2 shows the baseline to week 12 mean IOP reduction results comparing formulations described herein compared to topical administration of timolol solution according to the methods described in EXAMPLE 2.

FIG. 3 shows the mean difference in IOP values at weeks 2, 6 and 12 within a treatment cycle according to the methods described in EXAMPLE 2.

FIG. 4 shows the use of rescue medication after third cycle administration, according to example administration methods disclosed herein, comparing implant formulations described herein containing 10 lag or 15 lag against timolol solution applied twice a day according to the methods described in EXAMPLE 2.

### DESCRIPTION

### Definitions

For the purposes of this description, we use the following terms as defined in this section, unless the context of the word indicates a different meaning.

As used herein, an “intraocular implant” and “intraocular drug delivery system” refers to a device or element that is structured, sized, or otherwise configured to be placed in an eye and that is capable of delivering a therapeutic level of a drug to the eye. Intraocular implants and drug delivery systems in accordance with the present disclosure are generally biocompatible with physiological conditions of an eye and do not cause adverse side effects or immunological reaction. The implants are preferably completely biodegradable. Intraocular implants may be placed in an eye without disrupting vision of the eye. Non-limiting examples include extruded filaments or rods comprising a biodegradable polymer matrix and an active agent, such as bimatoprost, associated with the polymer matrix, and having a diameter and cut to a length suitable for placement in an ocular region of the eye, such as the anterior chamber.

An “intracameral implant” is an intraocular implant that is structured, sized, or otherwise configured to be placed in the anterior chamber of an eye. The anterior chamber of the eye refers to the fluid-filled space inside the eye between the iris and the innermost corneal surface (corneal endothelium). An intracameral implant will preferably fit into the anterior chamber angle, the junction of the front surface of the iris and back surface of the cornea, without contacting the corneal endothelium and thereby without causing corneal trauma, inflammation, or edema, or iris chaffing.

An “intravitreal” implant is an intraocular implant that is sized for placement in the vitreous body of the eye.

As used herein, “associated with the biodegradable polymer matrix” can mean any one or more of mixed with, dispersed within, coupled to, covering, or surrounding. Usually, the prostamide is non-covalently associated with the polymer matrix and is dispersed within and/or throughout the matrix.

As used herein, an “ocular region” or “ocular site” refers generally to any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of ocular regions in the eye include the anterior chamber, the posterior chamber, the vitreous cavity, the vitreous body, the choroid, the suprachoroidal space, the conjunctiva, the subconjunctival space, the sub-tenon space, the episcleral space, the intracorneal space, the epicorneal space, the sclera, the pars plana, surgically-induced avascular regions, the macula, and the retina.

As used herein, an “ocular condition” is a disease, ailment or medical condition which affects or involves the eye or one of the parts or regions of the eye. An ocular condition may be classified as an anterior or posterior ocular condition. Broadly speaking the eye includes the eyeball and the tissues and fluids which constitute the eyeball, the periocular muscles (such as the oblique and rectus muscles) and the portion of the optic nerve which is within or adjacent to the eyeball. Examples of an ocular condition within the scope of this disclosure include elevated intraocular pressure, ocular hypertension, and glaucoma. Glaucoma in a patient may be further classified as open-angle glaucoma or angle-closure glaucoma. A patient may be specifically diagnosed with primary open-angle glaucoma.

An anterior ocular condition is a disease, ailment or condition which affects, or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the ciliary body, the posterior chamber, the lens or the lens capsule and blood vessels and nerve which vascularize or innervate an anterior ocular region or site. Glaucoma can also be considered to be an anterior ocular condition because a clinical goal of glaucoma treatment can be to reduce a hypertension of aqueous fluid in the anterior chamber of the eye (i.e. reduce intraocular pressure).

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site. Glaucoma can also be considered a posterior ocular condition because the therapeutic goal is to prevent the loss of or reduce the occurrence of loss of vision due to damage to or loss of retinal cells or optic nerve cells (i.e. neuroprotection).

Intraocular pressure refers to the fluid pressure in the eye and is determined by the difference in the rate of aqueous humor secretion and outflow. Approximately 90% of the aqueous humor secreted exits through the trabecular meshwork in the anterior chamber. Resistance to outflow can lead to elevated intraocular pressure. Some populations or patient groups with normal tension (i.e., normotensive) glaucoma may have an TOP of from about 11 to 21 mm Hg. Some patient groups or patients with elevated intraocular pressure or ocular hypertension may have an TOP of greater than 20 or 21 mm Hg, as measured with a tonometer. Implants of the present disclosure are expected to be capable of reducing intraocular pressure in both normotensive and hypertensive glaucoma patients.

The term “biodegradable polymer” and “biodegradable intraocular implant” refers to a polymer or intraocular implant that degrade in vivo, and wherein erosion of the polymer or implant over time occurs concurrent with or subsequent to release of the therapeutic agent. The terms “biodegradable” and “bioerodible” are equivalent and are used interchangeably herein. A biodegradable polymer may be a homopolymer, a copolymer, or a polymer comprising more than two different polymeric units. Examples of biodegradable polymers within the scope of this disclosure are poly(D,L-lactide) polymers and poly(D,L-lactide-co-glycolide) copolymers.

The terms “treat,” “treating,” or “treatment” as used herein, refers to reduction or resolution or prevention of an ocular condition, ocular injury or damage, or to promote healing of injured or damaged ocular tissue. A treatment may be effective to reduce at least one sign or symptom of the ocular condition or risk factor associated with an ocular condition.

The term “therapeutically effective amount” as used herein, refers to the level or amount of agent needed to treat an ocular condition, or reduce or prevent ocular injury or damage without causing significant negative or adverse side effects to the eye or a region of the eye.

“Active agent,” “drug,” “therapeutic agent,” “therapeutically active agent,” and “pharmaceutically active agent” refer to the chemical compound that produces a therapeutic effect in the patient to which it is administered and that can be used to treat the ocular condition in the patient. One example of a therapeutically active agent and therapeutic agent is bimatoprost. In preferred embodiments the therapeutic effect is an intraocular pressure (IOP)-lowering effect, which can be identified by applying the compound to an eye and evaluating whether the intraocular pressure decreases after application.

Unless further specified, a “patient” refers to a human subject or non-human mammal in need of treatment for the ocular condition. For example, a patient may be further classified as a human patient. The term “mammal” includes both human patients and non-human mammals. Non-limiting examples of non-human mammals that may be subjects for any of the presently disclosed methods can include horses, dogs, monkeys, pigs, rabbits, and the like.

The term “biocompatible” and “compatible” means compatible with living tissue or a living system. Biocompatible implants and polymers produce few or no toxic effects, are not injurious, or physiologically reactive with living tissue and do not cause an immunological reaction.

Those skilled in the art will appreciate the meaning of various terms of degree used herein. For example, as used herein in the context of referring to an amount (e.g., “about 6%”), the term “about” represents an amount close to and including the stated amount that still performs a desired function or achieves a desired result, e.g. “about 6%” can include 6% and amounts close to 6% that still perform a desired function or achieve a desired result. For example, the term “about” can refer to an amount that is within less than 10% of, within less than 5% of, within less that 0.1% of, or within less than 0.01% of the stated amount.

Prostamides are potent ocular hypotensive agents useful in the treatment of a number of various ocular hypertensive conditions such as glaucoma, elevated intraocular pressure, and other ocular hypertensive episodes, including post-surgical and post-laser ocular hypertensive episodes (1, 4). They belong to an ever-expanding family of prostaglandin F_{2α} C-1 amides (1-5). The biosynthesis and pharmacology of prostamides has been extensively described (1-3, 9). For example, naturally occurring prostamides, such as prostamide F_{2α}, are biosynthesized from anandamide by a pathway exclusively involving COX-2. COX-1 is not involved (1, 2, 15). Other commercially available prostaglandin analogs include travoprost and latanoprost.

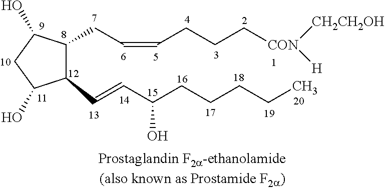

One prostamide that has found wide-spread use in ocular therapy is bimatoprost. Like other prostamides, bimatoprost exhibits no meaningful interaction with prostaglandin (PG) sensitive receptors (3, 10). Nevertheless, bimatoprost is a potent ocular anti-hypertensive agent and is highly effective for reducing elevated intraocular pressure in patients with open angle glaucoma or ocular hypertension (1, 6-8). Bimatoprost is typically prescribed for use by patients in the form of an ophthalmic solution known by the tradename LUMIGAN®. In the usual course of therapy, Patients apply one drop of LUMIGAN® solution once daily to the surface of the affected eye(s) to reduce elevated intraocular pressure. Bimatoprost is believed to decrease intraocular pressure (IOP) by increasing aqueous humor outflow through the uveoscleral pathway.

Glaucoma is generally a progressive disease of the eye characterized by progressive optic neuropathy with associated visual field loss. Glaucoma may be further associated with increased intraocular pressure. On the basis of its etiology, glaucoma has been classified as primary or secondary. Primary glaucoma in adults may be either open-angle glaucoma or acute or chronic angle-closure glaucoma. Secondary glaucoma results from pre-existing ocular diseases such as uveitis, intraocular tumor or an enlarged cataract.

The underlying causes of primary glaucoma are not yet known. Risk factors include high or elevated intraocular pressure, advanced age, and family history. Increased or elevated intraocular pressure is due to the obstruction of aqueous humor outflow. In primary open-angle glaucoma, the anterior chamber and its anatomic structures appear normal, but drainage of the aqueous humor is impeded. In acute or chronic angle-closure glaucoma, the anterior chamber is shallow, the filtration angle is narrowed, and the iris may obstruct the trabecular meshwork at the entrance of the canal of Schlemm. Dilation of the pupil may push the root of the iris forward against the angle, and may produce pupillary block and thus precipitate an acute attack. Eyes with narrow anterior chamber angles are predisposed to acute angle-closure glaucoma attacks of various degrees of severity.

Secondary glaucoma is caused by any interference with the flow of aqueous humor from the posterior chamber into the anterior chamber and subsequently, into the canal of Schlemm. Inflammatory disease of the anterior segment may prevent aqueous escape by causing complete posterior synechia in iris bombe and may obstruct movement of aqueous humor through the pupil leading to elevated intraocular pressure. Other common causes are intraocular tumors, enlarged cataracts, central retinal vein occlusion, trauma to the eye, operative procedures and intraocular hemorrhage. Considering all types together, glaucoma occurs in about 2% of all persons over the age of 40 and may be asymptomatic for years before progressing to noticeable peripheral visual loss followed by central vision loss.

Glaucoma can be considered to be potentially both an anterior and posterior ocular condition because a clinical goal of glaucoma treatment can be not only to reduce elevated intraocular pressure because of obstructed aqueous humor outflow from the anterior chamber, but to also prevent the loss of or reduce the occurrence of loss of vision due to damage to or loss of retinal cells or optic nerve cells (i.e., ganglion cells) in the posterior of the eye (i.e. neuroprotection). Clinical trials have shown that reducing TOP can help retard the progression of glaucoma and consistent TOP reduction is associated with reduced risks of developing and progressing optic nerve damage.

Patient non-adherence to topical therapy is one of the major challenges to preventing vision loss due to glaucoma. Patients that take no medication are at the highest risk of vision loss from glaucoma; however, patients that intermittently take their medications are also at risk since TOP fluctuation has also been identified as possible risk factor for progression in some patients.

Accordingly, sustained-release drug delivery systems, such as biodegradable intraocular implants, that can continuously deliver a therapeutically effective amount of an anti-hypertensive drug such as bimatoprost directly into the anterior chamber of the eye may help reduce patient dependence on topical ocular anti-hypertensive or other anti-glaucoma medications to control intraocular pressure and manage symptoms associated with glaucoma. Certain methods of administering such biodegradable intraocular methods can also improve the patient's outcomes, and minimize the number of treatments a patient may need to achieve a satisfactory lowering of intraocular pressure.

The present disclosure provides for improved methods for treatment of open angle glaucoma or ocular hypertension with surprisingly sustained reduction in intraocular pressure and/or without the need for topical ocular rescue medication.

The present disclosure provides for methods of treatment of open angle glaucoma or ocular hypertension including administration of a bimatoprost-containing biodegradable intraocular implant for reducing intraocular pressure (IOP) in an eye for at least 12 months. The implant may be effective for maintaining intraocular pressure in an eye at a reduced level (relative to the intraocular pressure in the eye before receiving the implant) for 12 months to 24 months, or for 24 months, or longer than 24 months after placement in the eye. The percent relative reduction in TOP in an eye after receiving the implant may vary, depending on the size of the implant (and therefore the drug load) and on the patient, but may be from 10-20%, 20-30%, or 10-50% below baseline TOP (the intraocular pressure in the eye before receiving the implant) and may, in some instances, remain at 20-30% below baseline TOP for at least 12 months, 12-24 months, or for 24 months or longer after implantation of a single implant.

The implant may be placed in an ocular region of an eye of a patient to reduce intraocular pressure in the eye and thereby to treat ocular hypertension and ocular conditions associated with elevated intraocular pressure, including glaucoma. The bimatoprost-containing implant described here is specifically sized and formulated for placement in the anterior chamber of the eye (also referred to herein as administration “intracamerally”). Anterior chamber angle widths may be graded according to the Shaffer System (Shaffer RN. (1960) “Primary glaucomas. Gonioscopy, ophthalmoscopy, and perimetry” Trans Am Acad Ophthalmol Otolaryngol. 64:112-127). Shaffer Grade 1 and Grade 2 angles may be considered narrow. It may be preferable to treat patients that have either a Shaffer Grade 1 or Grade 2 angle by placing the implant in the vitreous body of the eye rather than the anterior chamber to reduce the chance of corneal toxicity. Patients with open angles, such as patients with Shaffer Grade 3 and 4 angles, may be candidates for either an intracameral implant or an intravitreal implant.

The biodegradable polymer matrix of an implant according to this disclosure may release a prostamide at a rate to sustain release of a therapeutically effective amount of the prostamide from the implant for a period of two months from a time in which the implant is placed in an ocular region of an eye. In some instances, the implant may be effective for reducing intraocular pressure in an eye for at least 12 months, or for 12 to 24 months, or 24 months or longer after placement of the implant in an eye. The implant is designed specifically for placement in the anterior chamber of the eye, but may be suitable for placement in other ocular regions to treat conditions such as glaucoma and ocular hypertension, or to generally reduce IOP in an eye. Accordingly, an implant according to this disclosure may, for example, be placed in the anterior chamber.

### Implants

As set forth above, an implant formulation according to this disclosure may contain bimatoprost or other prostamide. In some embodiments, the prostamide contained by the implant comprises a compound having the Formula (I)

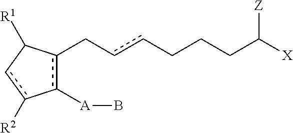

wherein the dashed bonds represent a single or double bond which can be in the cis or trans configuration, A is an alkylene or alkenylene radical having from two to six carbon atoms, which radical may be interrupted by one or more oxide radicals and substituted with one or more hydroxy, oxo, alkyloxy or alkylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R4)2 wherein R4 is independently selected from the group consisting of hydrogen and a lower alkyl radical having from one to six carbon atoms; Z is ═O; one of R1 and R2 is ═O, —OH or a —O(CO)R6 group, and the other one is —OH or —O(CO)R6, or R1 is ═O and R2 is H, wherein R6 is a saturated or unsaturated acyclic hydrocarbon group having from 1 to about 20 carbon atoms, or —(CH₂)mR7 wherein m is 0 or an integer of from 1 to 10, and R7 is cycloalkyl radical, having from three to seven carbon atoms, or a hydrocarbyl aryl or heteroaryl radical, as defined above.

In a more specific embodiment the prostamide contained by the implant is bimatoprost, which has the following chemical structure:

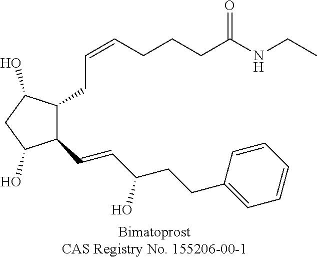 [SMILES: CCNC(=O)CCC/C=C\C[C@@H]1[C@@H](/C=C/[C@@H](O)CCc2ccccc2)[C@H](O)C[C@@H]1O]

Other examples of prostamides (prostaglandin F2α amides) may include, but are not limited to, the prostaglandin F2α amides described in Woodward et al. (2008) “Prostamides (prostaglandin ethanolamides) and their pharmacology” British J. Pharmacology 153:410-419; and Schuster et al. (2000) “Synthetic modification of prostaglandin F2α indicates different structural determinants for binding to the prostaglandin F receptor versus the prostaglandin transporter” Molecular Pharmacology 58:1511-1516; and the prostaglandin F2α amides described in U.S. Pat. Nos. 5,688,819 and 5,834,498, which are herein incorporated by reference.

In general, an intraocular implant in accordance with this disclosure comprises or consists of bimatoprost as the active agent, a biodegradable polymer matrix, and optionally a polyethylene glycol. The bimatoprost (or other prostamide) may comprise from 5% to 90% by weight of the implant, or from 5% to 30% by weight of the implant, or from 18-22% by weight of the implant, but is preferably 20% by weight of the implant. The biodegradable polymer matrix will generally comprise a mixture of at least three different biodegradable polymers independently selected from the group consisting of poly(D,L-lactide) (PLA) polymers and poly(D,L-lactide-co-glycolide) (PLGA) polymers. For example, the biodegradable polymer matrix may comprise or consist of first, second, and third biodegradable polymers that differ one from the other by their repeating unit, inherent viscosity, or end-group, or any combination thereof. In some instances, the biodegradable polymer matrix according to the present disclosure may comprise first, second, third, and fourth biodegradable polymers independently selected from the group consisting of poly(D,L-lactide) (PLA) polymers and poly(D,L-lactide-co-glycolide) (PLGA) polymers, wherein the first, second, third, and fourth polymers differ one from the other by their repeating unit, inherent viscosity, or end-group, or any combination thereof. Depending on the chain terminating agent used during the synthesis of the polymer, a PLA or PLGA polymer may have a free carboxylic acid end group or alkyl ester end group, and may be referred to herein as an acid-end or ester-end (or ester-capped) PLA or PLGA polymer, respectively.

In one embodiment, the biodegradable polymer matrix comprises or consists of first, second, and third biodegradable polymers, wherein the first biodegradable polymer is an ester-end poly(D,L-lactide) polymer having an inherent viscosity of 0.25-0.35 dl/g, the second polymer is an acid-end poly(D,L-lactide) polymer having an inherent viscosity of 0.16-0.24 dl/g, and the third polymer is a ester-end poly(D,L-lactide-co-glycolide) polymer having a D,L-lactide:glycolide molar ratio of from 73:27 to 77:23, or about 75:25, and an inherent viscosity of 0.16-0.24 dl/g, where the inherent viscosity of each polymer is determined for a 0.1% w/v solution of the polymer in chloroform at 25° C.

The prostamide contained by the implant may be uniformly or non-uniformly distributed throughout the biodegradable polymer matrix. The prostamide may be dispersed within the biodegradable polymer matrix.

As stated above, the implant may further comprise polyethylene glycol. The polyethylene glycol contained by the implant may have an average molecular weight of from 3000 to 20,000 g/mol. In one embodiment the implant contains polyethylene glycol 3350 (PEG 3350). The polyethylene glycol will generally be associated with the biodegradable polymer matrix. For example, the polyethylene glycol may be dispersed within the biodegradable polymer matrix.

The polyethylene glycol (PEG) in any of the foregoing embodiments may have an average molecular mass of from 3,000 to 20,000 g/mol. In preferred embodiments the polyethylene glycol in the implant is PEG 3350. For example, one embodiment provides for a biodegradable intraocular implant comprising 20% by weight (w/w) bimatoprost, 20% by weight R203 S, 15% by weight R202H, 40% by weight RG752S, and 5% by weight polyethylene glycol 3350 (Formulation 2). More generally, the implant can comprise 18-22% by weight (w/w) bimatoprost, 18-22% by weight R203S, 13.5-16.5% by weight R202H, 36-44% by weight RG752S, and 3.5-6.5% by weight polyethylene glycol.

In this regard, one embodiment is a biodegradable intraocular implant for reducing intraocular pressure or ocular hypertension in a patient, the implant comprising a biodegradable polymer matrix and a prostamide as the active agent associated with the biodegradable polymer matrix, the biodegradable polymer matrix comprising or consisting of
a) R203S, which is an ester end poly(D,L-lactide) having an inherent viscosity of 0.25-0.35 dl/g;
b) R202H, which is an acid end poly(D,L-lactide) having an inherent viscosity of 0.16-0.24 dl/g;
c) RG752S, which is an ester end poly(D,L-lactide-co-glycolide) having a D,L-lactide:glycolide molar ratio of about 75:25 and an inherent viscosity of 0.16-0.24 dl/g; and
d) polyethylene glycol 3350;
wherein the prostamide comprises 20% of the implant by weight, the ester end poly(D,L-lactide) comprises 20% of the implant by weight, the acid end poly(D,L-lactide) comprises 15% of the implant by weight, the ester end poly(D,L-lactide-co-glycolide) comprises 40% of the implant by weight, and wherein the polyethylene glycol (PEG) 3350 comprises 5% of the implant by weight, wherein the inherent viscosities for each of the poly(D,L-lactide) and poly(D,L-lactide-co-glycolide) polymers are measured for a 0.1% solution of the polymer in chloroform at 25° C.

In some embodiments, the prostamide is a compound having Formula I. In one embodiment the prostamide is bimatoprost.

Examples of intraocular implants for use in a method of treating an ocular condition in accordance with this disclosure include those set forth in Tables 1 and 2, below. For example an intraocular implant for reducing intraocular pressure and ocular hypertension in a patient may comprise 20% by weight bimatoprost, 15% by weight R203S, 20% by weight RG858S, 40% by weight RG752S, and 5% by weight polyethylene glycol 3350. RG858S is an ester end poly(D,L-lactide-co-glycolide) having an inherent viscosity of 1.3-1.7 dl/g and a D,L-lactide to glycolide ratio of 83:17 to 87:13, or about 85:15.

Another embodiment is a biodegradable intraocular implant for treating an ocular condition in an eye of a patient, the implant comprising 18-22% by weight (w/w) bimatoprost, 18-22% by weight R203S, 13.5-16.5% by weight R202H, 36-44% by weight RG752S, and 3.5-6.5% by weight polyethylene glycol.

An additional embodiment is a biodegradable intraocular implant for treating an ocular condition in an eye of a patient, the implant comprising 20% by weight (w/w) bimatoprost, 20% by weight R203S, 15% by weight R202H, 40% by weight RG752S, and 5% by weight polyethylene glycol.

Another embodiment is a biodegradable intraocular implant for treating an ocular condition in an eye of a patient, the implant comprising 20% by weight (w/w) bimatoprost, 15% by weight RG858S, 35% by weight RG752S, 15% by weight RG755 S, and 15% by weight RG502S. RG755 S is a poly(D,L-lactide-co-glycolide) having an ester end group, an inherent viscosity of about 0.50-0.70 dl/g (as measured for a 0.1% solution in chloroform at 25° C.), and a D,L-lactide:glycolide molar ratio of 73:27 to 77:23, or about 75:25. RG502S is a poly(D,L-lactide-co-glycolide) having an ester end group, an inherent viscosity of 0.16-0.24 dl/g (as measured for a 0.1% solution in chloroform at 25° C.), and a D,L-lactide:glycolide ratio of 48:52 to 52:48, or about 50:50.

Another embodiment is a biodegradable intraocular implant for treating an ocular condition in an eye of a patient, the implant comprising 20% by weight (w/w) bimatoprost, 30% by weight RG858S, 40% by weight RG752S, 5% by weight RG502, and 5% by weight RG502H. RG502H is a poly(D,L-lactide-co-glycolide) having an acid end group, an inherent viscosity of 0.16-0.24 dl/g (as measured for a 0.1% solution in chloroform at 25° C.), and a D,L-lactide:glycolide ratio of about 50:50 (such as for example RG502H).

Another embodiment is a biodegradable intraocular implant comprising 20% by weight (w/w) bimatoprost, 20% by weight RG752S, 50% by weight RG755S, 5% by weight RG502, and 5% by weight RG502H.

Another embodiment is a biodegradable intraocular implant comprising 20% by weight (w/w) bimatoprost, 25% by weight RG752S, 50% by weight RG755S, and 5% by weight RG502. Another embodiment is a biodegradable intraocular implant comprising 20% by weight (w/w) bimatoprost, 30% by weight RG752S, 20% by weight RG502, and 30% by weight RG858S

The present disclosure also provides for methods of making a biodegradable intraocular prostamide-containing implant that will release a therapeutically effective amount of a prostamide in an eye for at least 60 days (two months). The method generally comprises combining a prostamide, at least three biodegradable polymers, and optionally a polyethylene glycol to form a combination of components, blending the combination to form a blended mixture, heating the blended mixture, then extruding the heated mixture to form a filament, and then cutting the filament to form an implant suitable for placement in an ocular region of an eye of a patient. For example, the implant may be cut to a length suitable (sized) for placement in the anterior chamber or vitreous body of the eye of the patient. Each of the components may be combined as dry powders or as dry solids. The blending step may therefore comprise dry powder blending. The at least three biodegradable polymers may be selected from the group consisting of poly(D,L-lactide) (PLA) polymers and poly(D,L-lactide-co-glycolide) (PLGA) polymers. For example, the at least three biodegradable polymers may consist of first, second, and third biodegradable polymers that differ one from the other by their repeating unit, inherent viscosity, and/or end-group. In some instances, the at least three biodegradable polymers may consist of first, second, third, and fourth biodegradable polymers that differ one from the other. The first, second, third, and, optionally fourth biodegradable polymers may be selected from acid-end and ester-end PLA and PLGA polymers. For example, the first, second, third, and optionally fourth biodegradable polymers used to make the implants according to the method described above may be selected from the group consisting of RESOMER® Biodegradable Polymers R203S, R202H, RG502, RG502H, RG752S, RG755S, and RG858S, wherein RG502 is a poly(D,L-lactide-co-glycolide) having an ester end group and an inherent viscosity of 0.16-0.24 dl/g and a D,L-lactide:glycolide ratio of about 50:50, RG502H is a poly(D,L-lactide-co-glycolide) having an acid end group and an inherent viscosity of 0.16-0.24 dl/g, and a D,L-lactide:glycolide ratio of about 50:50, and RG755S is an ester end poly(D,L-lactide-co-glycolide) having an inherent viscosity of 0.50-0.70 dl/g and a D,L-lactide:glycolide ratio of about 75:25. In some embodiments the polyethylene glycol is polyethylene glycol 3350 (PEG 3350).

Thus, one embodiment is a method for making a biodegradable intraocular implant comprising mixing a prostamide with a) an ester end poly(D,L-lactide) having an inherent viscosity of 0.25-0.35 dl/g, b) an acid end poly(D,L-lactide) having an inherent viscosity of 0.16-0.24 dl/g, and c) an ester end poly(D,L-lactide-co-glycolide) having an inherent viscosity of 0.16-0.24 dl/g and a D,L-lactide to glycolide molar ratio of about 75:25, and with polyethylene glycol 3350, extruding the mixture to form a filament, followed by cutting the filament to length suitable for placement in the anterior chamber or vitreous body of an eye to thereby form an intraocular implant, wherein the prostamide comprises about 20% of the implant by weight, the ester end poly(D,L-lactide) comprises about 20% of the implant by weight, the acid end poly(D,L-lactide) comprises about 15% of the implant by weight, the ester end poly(D,L-lactide-co-glycolide) comprises about 40% of the implant by weight, and the polyethylene glycol 3350 comprises about 5% of the implant by weight. Unless otherwise specified the inherent viscosity for the PLA and PLGA polymers set forth herein is measured for a 0.1% solution of the polymer in chloroform at 25° C.

One example of an intraocular implant (i.e., drug delivery system) is an extruded biodegradable intraocular implant sized for implantation in the anterior chamber of an eye, the implant comprising or consisting of 20% by weight (w/w) bimatoprost, 5% by weight PEG 3350, 20% by weight R203S, which is an ester-end poly(D,L-lactide) polymer having an inherent viscosity of 0.25-0.35 dl/g, 15% by weight R202H, which is an acid-end poly(D,L-lactide) polymer having an inherent viscosity of 0.16-0.24 dl/g, and 40% by weight RG752S, which is an ester-end poly(D,L-lactide-co-glycolide) polymer having a D,L-lactide:glycolide molar ratio of about 75:25 and an inherent viscosity of 0.16-0.24 dl/g, wherein the inherent viscosity of each polymer is measured for a 0.1% w/v solution in chloroform at 25° C. The implant may sustain release of a therapeutically effective amount of the bimatoprost into an eye for a period of two months or longer.

In some embodiments the intraocular implant is sized and formulated for placement in the anterior chamber of the eye (i.e., for intracameral administration). An implant sized for placement in the anterior chamber of an eye and capable of delivering a therapeutically effective amount of bimatoprost to the mammalian eye for an extended period according to this disclosure is generally from 20 μg to 200 μg in total weight, from 0.5 to about 3.0 mm in length, and from 0.1 to 0.5 mm in diameter (or other smallest dimension as may be appropriate for non-cylindrical implants). In some embodiments, an implant sized for placement in the anterior chamber (an intracameral implant) may weigh (therefore have a total weight) from about 30 to about 150 μg and contain from about 6 μg to about 30 μg of bimatoprost or other prostamide. In a preferred embodiment, the intracameral implant has a total weight of from 30 to 150 μg and is 150 μm to 300 μm in diameter and 0.5 mm to 2.5 mm in length. In a more preferred embodiment the biodegradable intracameral implant according to this disclosure has a total weight of 30 μg to 100 μg and is 150 μm to 300 μm in diameter and 0.5 mm to 2.5 mm in length. In some embodiments, the implant is about 150 to about 300 μm in diameter or width, about 1.0 mm to about 2.5 mm in length, and about 30 μg to about 100 μg in total weight. In some embodiments, the implant is 150 to about 300 μm in diameter or width, 1.0 mm to 2.5 mm in length, and 30 μg to 75 μg, or 30 to 90 μg in total weight. The implant may be an extruded implant (i.e., the implant may be produced by an extrusion process). In some embodiments, the implant is formed by an extrusion process and is 150 to 300 μm in diameter or width, 0.50 to 2.5 mm in length, and 30 to 100 μg in total weight.

Thus, an intracameral implant according to this disclosure may have a total weight of from 20-120 μg, 30-100 μg, 30-90 μg, 30-75 μg, or 30-50 μg. Non-limiting examples include extruded implants containing about 6 μg, 10 μg, 15 μg, or 20 μg (±5%) bimatoprost and having a total weight of about 30 μg, 50 μg, 75 μg, or 100 μg (±5%), respectively. In certain forms the extruded implant may have a diameter of about 200 μm or 250 μm (±5%) (before placement in the eye or other liquid or fluid environment) and a length of about 2.3 mm, 1.5 mm, or 1.0 mm (±5%). Preferably, the implant can be received in, and injected into the eye through, a 27, 28, or 30 gauge ultra-thin-wall needle. Small diameter needles such as these may be desirable for delivery of implants into the anterior chamber of the eye. Implants of the particular size described here may have the additional advantage of fitting within the anterior chamber angle of the eye without causing corneal trauma (e.g. edema) and without chaffing the iris. In one embodiment the intracameral implant is about 200 μm to about 300 μm in diameter, and about 1.0 to about 2.3 mm in length. An implant sized for placement in the anterior chamber of an eye according to this disclosure and according to any of the foregoing embodiments can comprise 20% (w/w) bimatoprost, 20% (w/w) R2035, 15% (w/w) R202H, 40% (w/w) RG752S, and 5% (w/w) polyethylene glycol (PEG) 3350. Implants are sized and formulated for placement in the anterior chamber in accordance with this disclosure so as to avoid contact with the corneal endothelium (i.e., so that the implant does not contact the corneal endothelium) after placement in the anterior chamber of an eye. Contact with the corneal endothelium may result in a loss of corneal endothelial cells (density reduction) and onset of corneal edema. The risk for such adverse effects generally rises with increasing size of the implant. With larger implants there is a greater likelihood of contact with the corneal endothelium, e.g., by touching the endothelium anterior to Schwalbe's line.

One embodiment is an extruded biodegradable intraocular implant according to this disclosure that is sized for placement in the anterior chamber of the eye, whereby the implant is 150 to 300 μm in diameter, 0.50 to 3 mm in length, and 25 to 100 μg in total weight. Another embodiment is an extruded biodegradable intraocular implant according to this disclosure that is sized for placement in the anterior chamber of the eye, whereby the implant is 150 to 250 μm (±5%) in diameter, 0.75 to 2 mm in length, and 50 to 75 μg in total weight. The implant according to either embodiment will usually comprise 20% by weight bimatoprost as the active agent in association with a biodegradable polymer matrix comprising or consisting of i) an ester-end poly(D,L-lactide), ii) an acid-end poly(D,L-lactide), and iii) an ester-end poly(D,L-lactide-co-glycolide) having a D,L-lactide:glycolide ratio of about 75:25 and an inherent viscosity of 0.16-0.24 dl/g, wherein the inherent viscosity is measured for a 0.1% solution of the polymer in chloroform at 25° C. In a more specific embodiment, the ester end poly(D,L-lactide) has an inherent viscosity of 0.25-0.35 dl/g and the acid-end poly(D,L-lactide) has an inherent viscosity of 0.16-0.24 dl/g.

A therapeutically effective amount of bimatoprost for reducing intraocular pressure in an eye of a patient may correspond to a bimatoprost release rate in the eye of about 50 to 500 ng/day. An implant according to Formulation 2, for example (Table 1), with a total weight of about 25 μg and comprising about 20% by weight bimatoprost (i.e., about 5 μg of bimatoprost) may release approximately 50 ng of bimatoprost per day following placement in the eye. A Formulation 2 implant having a total weight of about 250 μg and comprising about 50 μg of bimatoprost may release approximately 500 ng of bimatoprost per day following placement in the eye.

The prostamide component of the implant may be in a particulate or powder form and it may be entrapped by, embedded within, or distributed uniformly or non-uniformly throughout the biodegradable polymer matrix. In the presently disclosed implants, the prostamide will usually comprise about 20% of the implant on a weight to weight (w/w) basis. In other words, the prostamide will constitute about 20% of the implant by weight. More generally, the prostamide can comprise (i.e., be present in an amount of or constitute) 18% and 22% of the implant by weight.

The intraocular implants described here comprise a mixture of at least three different biodegradable polymers selected from the group consisting of poly(D,L-lactide) (PLA) polymers and poly(D,L-lactide-co-glycolide) (PLGA) polymers. Differences between the three polymers may be with regard to the end group, inherent viscosity, or repeating unit, or any combination thereof.

If used, a poly(D,L-lactide-co-glycolide) comprises one or more blocks of D,L-lactide repeat units (x) and one or more blocks of glycolide repeat units (y), where the size and number of the respective blocks may vary. The molar percent of each repeat unit in a poly(lactide-co-glycolide) (PLGA) copolymer may be independently 0-100%, 50-50%, about 15-85%, about 25-75%, or about 35-65%. In some embodiments, the D,L-lactide may be about 50% to about 85% of the PLGA polymer on a molar basis. The balance of the polymer may essentially be the glycolide repeat units. For example, the glycolide may be about 15% to about 50% of the PLGA polymer on a molar basis.

The present disclosure provides for a biodegradable intraocular implant for reducing intraocular pressure (TOP) in an eye comprising a biodegradable polymer matrix, polyethylene glycol 3350, and a prostamide as the active agent, wherein the prostamide and polyethylene glycol 3350 are associated with the biodegradable polymer matrix, which comprises an ester end poly(D,L-lactide) having an inherent viscosity of 0.25-0.35 dl/g, an acid end poly(D,L-lactide) having an inherent viscosity of 0.16-0.24 dl/g, and an ester end poly(D,L-lactide-co-glycolide) having an inherent viscosity of 0.16-0.24 dl/g and a D,L-lactide to glycolide molar ratio of about 75:25, wherein the prostamide constitutes 18 to 22% of the implant by weight, the ester end poly(D,L-lactide) constitutes 18 to 22% of the implant by weight, the acid end poly(D,L-lactide) constitutes 13.5 to 16.5% of the implant by weight, the ester end poly(D,L-lactide-co-glycolide) constitutes 36 to 44% of the implant by weight, and wherein the polyethylene glycol 3350 constitutes 3.5 to 6.5% of the implant by weight, wherein the inherent viscosity of each of the poly(D,L-lactide) and poly(D,L-lactide-co-glycolide) polymers is determined for a 0.1% solution of the polymer in chloroform at 25° C. In a specific embodiment the prostamide constitutes 20% of the implant by weight, the ester end poly(D,L-lactide) constitutes 20% of the implant by weight, the acid end poly(D,L-lactide) constitutes 15% of the implant by weight, the ester end poly(D,L-lactide-co-glycolide) constitutes 40% of the implant by weight, and the polyethylene glycol 3350 constitutes 5% of the implant by weight.

In some embodiments the implant defined above is rod-shaped and is formed by a hot-melt extrusion process such that the formed implant is 150 to 300 μm in diameter or width, 0.50 to 2.5 mm in length, and 30 to 100 μg in total weight, whereby the implant does not contact the corneal endothelium after placement in the anterior chamber of an eye.

Certain example formulations are described below:

TABLE 1
Bimatoprost Containing Sustained Delivery Formulations (1-5) for the
production of extruded intracameral implants

| | | Polymer, excipient % w/w | | | | |
|---|---|---|---|---|---|---|
| Formulation | Bimatoprost | | | | | PEG |
| No. | % w/w | R203S | R202H | RG752S | RG858S | 3350 |
| 1 | 20 | 45 | 10 | 20 | | 5 |
| 2 | 20 | 20 | 15 | 40 | | 5 |
| 5 | 20 | 15 | | 40 | 20 | 5 |

TABLE 2
Bimatoprost-containing Sustained Delivery Formulations (6-8) for the
production of extruded intracameral implants

| Formulation | Bimatoprost | Polymer % w/w | | | | |
|---|---|---|---|---|---|---|
| No. | % w/w | RG752S | RG755S | RG502 | RG502H | RG858S |
| 3 | 20 | 35 | 15 | 15 | | 15 |
| 4 | 20 | 40 | | 5 | 5 | 30 |
| 6 | 20 | 20 | 50 | 5 | 5 | |
| 7 | 20 | 25 | 50 | 5 | | |
| 8 | 20 | 30 | | 20 | | 30 |

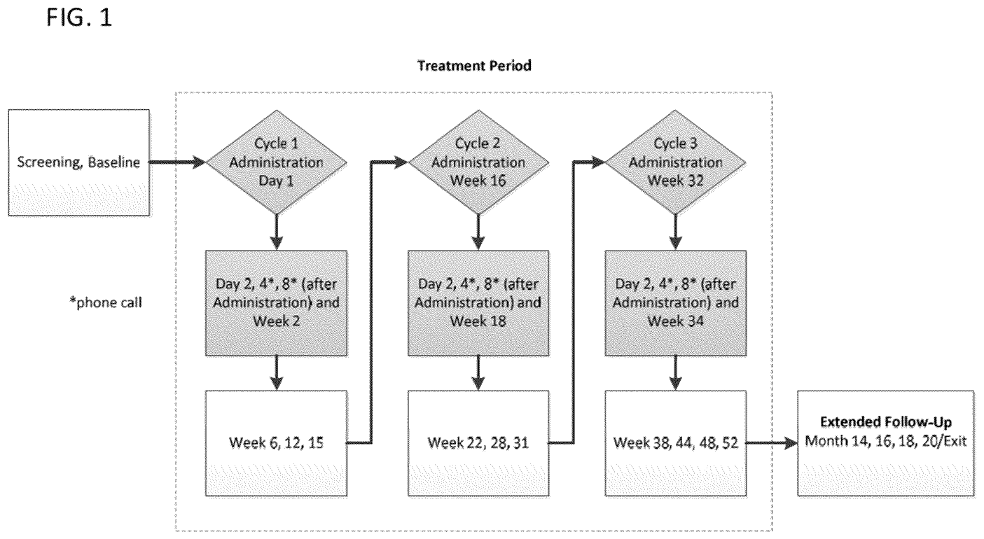

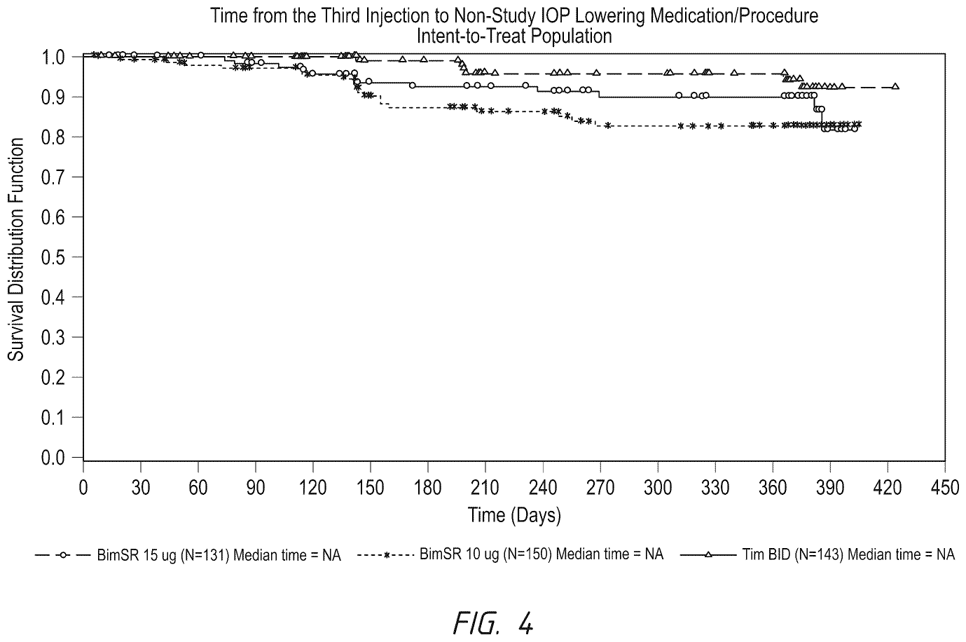

As described in Table 3 of US Patent Application Publication No. 2015-0118279, most of the example formulations only exhibited an in vitro drug release profile of about 60 days or two months or fewer than 100 days as shown in FIG. 1 of the US Patent Application Publication. In the in vivo beagle dog studies shown in Table 4, and FIG. 4 of US Patent Application Publication No. 2015-0118279, there was statistically significant difference at 4 months, and the IOP reduction was only measured out to six months.

### Methods of Treatment

The intraocular implants according to this disclosure can be effective for reducing intraocular pressure in either a normotensive or hypertensive eye for an extended period. In some embodiments of the present methods, a patient may have normal tension glaucoma (NTG), with an intraocular pressure ranging from 11 to 21 mm Hg. Such patients may require even lower eye pressures to reduce the risk of progressive optic nerve damage and visual field loss, and may benefit from the intraocular administration of an implant according to this disclosure. Thus, an implant according to this disclosure may be effective for treating glaucoma in all its forms, including glaucoma characterized by elevated intraocular pressure, as well as low-tension or normal-tension glaucoma, since these patients, too, may potentially benefit from a further reduction in intraocular pressure.

The implant may be effective for reducing intraocular pressure in an eye by 10-20%, 20-30%, and possibly by 30-40% or more (with higher drug release rates), relative to the intraocular pressure (IOP) in the eye before receiving the implant, for 12 months or more, 16 months or more, or 24 months, or 24 months or more after placement of the implant in the eye. Such implants may further be effective for reducing the risk of developing, delaying the onset of, or slowing the progression of glaucomatous damage in an eye of a patient. Glaucomatous damage in the eye may include damage to the function and/or structure of the optic nerve and ganglion cell death, which can lead to loss of peripheral visual fields and eventually central vision loss leading to total blindness. Elevated IOP presents a major risk factor for glaucomatous field loss.

Accordingly, the presently described implants may be effective for treating a patient suffering from or diagnosed with an ocular condition selected from glaucoma, open angle glaucoma, primary open-angle glaucoma, angle-closure glaucoma (sometimes referred to as closed-angle glaucoma), normal-tension glaucoma, low-tension glaucoma, pseudoexfoliative glaucoma, developmental glaucoma, or pigmentary glaucoma. One or more of the present implants may also be useful for reducing and thereby treating ocular hypertension or elevated intraocular pressure. For example, an implant according to this disclosure may be effective for reducing intraocular pressure in a patient with open-angle glaucoma, angle-closure glaucoma, or ocular hypertension. The patient can be a human or non-human mammal. The method will generally comprise the step of placing the implant in an ocular of the eye affected by the ocular condition.

Because of their ability to release a therapeutically effective amount of bimatoprost for an extended period (e.g., 60 days or longer), implants in accordance with this disclosure are expected to be capable of reducing intraocular pressure in a patient for long periods (e.g., for 12 months or more or 24 months or more) without the need for frequent intraocular injections or regular instillation of eye drops to the ocular surface as may be necessary with topical therapy. Accordingly, in some forms, the implants described here are used as monotherapy (i.e. used alone to control the TOP without the use of adjunctive antihypertensive eye drops) to reduce intraocular pressure in a patient and thereby treat an ocular condition as described herein. Nevertheless, an implant in accordance with this disclosure can, if desired, be used in dual therapy in conjunction with the same or different therapeutic agent that is applied topically.

The implant will preferably deliver a therapeutically effective dose of the prostamide to the eye(s) for at least two months after placement in the eye, and will reduce the ocular condition, or at least one sign or symptom, or risk factor associated with the ocular condition, for at least 1 month, or for at least 2, or 4 months following placement of the implant in the anterior chamber of the eye. If desired, more than one implant can be placed in the eye. For example, two implants may be placed in the anterior chamber or vitreous body of the eye to deliver a larger dose of the prostamide. For example, in one method an eye may be dosed with 20 μg of bimatoprost, by placing two 50-μg implants (each containing 20% bimatoprost by weight) in the anterior chamber of the eye simultaneously rather than using a single 100-μg implant. Using two smaller implants may possibly improve the tolerability of the implants in the eye and further reduce the risk of an implant contacting the corneal endothelium, thereby lessening or altogether eliminating the chance that the eye will experience a loss of corneal endothelial cell density and onset of corneal edema.

One embodiment is a method for reducing intraocular pressure in an eye of a mammal, the method comprising placing a biodegradable intraocular implant according to this disclosure in an eye of the mammal, whereby the implant provides a prostamide to the eye in an amount effective for reducing intraocular pressure in the eye. In some forms of this method the mammal is a human patient that has elevated intraocular pressure, ocular hypertension, or glaucoma, and the implant is placed in the anterior chamber of the affected eye(s) of the patient. According to some embodiments, the method is effective for the lowering of intraocular pressure (IOP) in patients with open angle glaucoma or ocular hypertension. In other embodiments, the methods are effective for the lowering of IOP in patients with open angle glaucoma. In some embodiments the methods are therapeutically effective for the lowering of IOP in patients with open angle glaucoma or ocular hypertension who are inadequately managed with topical IOP-lowering medication (e.g., due to intolerance or nonadherence) or are unsuitable for topical therapy. In some embodiments the methods are therapeutically effective for the lowering of IOP in patients with open angle glaucoma who are inadequately managed with topical IOP-lowering medication (e.g., due to intolerance or nonadherence) or are unsuitable for topical therapy. The implant may be effective for reducing intraocular pressure in the eye for at least two months after placement in the anterior chamber of the eye. In some instances, the implant may reduce intraocular pressure in the eye for greater than 12 months after placement of the implant in the eye. In some embodiments, a single implant may reduce intraocular pressure for between 12 and 24 months. In one embodiment the prostamide provided by the implant is bimatoprost. Preferably, the implant is sized and formulated for placement in the anterior chamber of the eye and does not contact and/or does not injure the corneal endothelium after placement in the anterior chamber of an eye, such as for example a human eye. Eliminating contact between the implant and the corneal endothelium may reduce the risk of corneal endothelial cell density reduction and onset of corneal edema in the eye.

The present disclosure also provides for a method for reducing or lowering intraocular pressure in a patient, the method comprising placing a biodegradable intraocular implant in an eye of the patient, thereby reducing intraocular pressure in the eye for an extended period such as, for example, for at least one month, two months, or for at least four months. In some instances, the patient may have open-angle glaucoma, or more specifically primary open-angle glaucoma, and/or ocular hypertension. The implant used in the method can be any of the prostamide-containing implants described herein. In a preferred embodiment, the method comprises placing an extruded intraocular implant comprising Formulation 2 in an eye of the patient. The implant can be placed in the anterior chamber, vitreous body, or posterior chamber of the eye, for example. In some instances, the implant may be specifically placed in the anterior chamber angle (iridocorneal angle) of the eye, and even more specifically in the inferior iridocorneal angle of the eye.

An extended duration of IOP-lowering effect may also be observed with other dosing regimens of an intraocular implant as described herein to treat a patient with open-angle glaucoma or ocular hypertension. For example, a patient may receive 1 or 2 or 3 or 4 or 5 or 6 or 7 or 8 total implants over a treatment duration, with a single intracameral implant injected into the patient's anterior chamber once every 3 months (about 12 weeks) or 4 months (about 16 weeks) or 5 months (about 20 weeks) or 6 (about 24 weeks) or 7 months (about 28 weeks) or 8 months (about 32 weeks) or 9 months (about 36 weeks) or 10 months (about 40 weeks) or 11 months (about 44 weeks) or 12 months (about 48 weeks) and experience an increased duration of IOP-lowering effect and/or amount of time without the need for a rescue medication for reduction of IOP (e.g., prostaglandin analog or prostamide—containing eye drops such as latanoprost, travoprost, or bimatoprost). The duration of IOP-lowing effect or amount of time without the need for a rescue medication after such dosing regimen may be 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, or more than 24 months. In some embodiments, the duration of IOP-lowing effect or amount of time without the need for a rescue medication can be in the range of 12 months to 24 months, over 12 months to 15 months, 13 months to 24 months, over 12 months to 24 months, over 12 months to 16 months, over 12 months to 20 months, over 16 months to 24 months, and the like. In some embodiments, the duration of IOP-lowing effect or amount of time without the need for a rescue medication can be in the range of 12 months to 24 months, over 12 months to 15 months, 13 months to 24 months, over 12 months to 24 months, over 12 months to 16 months, over 12 months to 20 months, over 16 months to 24 months, after receipt of the final implant over the treatment duration.

According to an embodiment, a patient having open angle glaucoma or ocular hypertension may receive a first intraocular biodegradable implant containing bimatoprost injected into a patient's anterior chamber at day 1, then a second implant containing bimatoprost injected into a patient's anterior chamber at week 16, then a final bimatoprost implant injected into a patient's anterior chamber at week 32. According to such embodiments, a patient with open angle glaucoma or ocular hypertension may receive an implant containing 10 μg of bimatoprost or 15 μg of bimatoprost, at day 1, week 16, and week 32. In some embodiments, a patient receives a first intraocular implant containing bimatoprost injected into a patient's anterior chamber at day 1, then a final implant containing bimatoprost at week 16 and no further implants. In some embodiments, a patient in need thereof only receives a single and final implant. In such methods, the sustained IOP-reduction effect can be observed for a period of time of 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, and the like after the injection of the final implant. In some embodiments, a patient having open angle glaucoma or ocular hypertension demonstrates a reduction of IOP of about 20-30% over the treatment duration according to the methods described above. In some embodiments, a patient having open angle glaucoma or ocular hypertension demonstrates a reduction of IOP of about 30% over the treatment duration according to the methods described above.

In one embodiment, the implant is placed in the eye(s) using an intraocular delivery apparatus, the apparatus comprising an elongate housing and a cannula extending longitudinally from the housing, the cannula having a proximal end and a distal sharp end and having a lumen extending therethrough, the lumen having an inner diameter sufficient to receive the implant and permit passage of the implant through the lumen and into the eye of the patient. The apparatus may further comprise a push rod or plunger operably connected with a user-actuated linkage for ejecting the implant through the lumen into the eye.

Another embodiment includes the use of an apparatus for delivering a biodegradable intraocular implant into the eye of a patient, the apparatus comprising an intraocular implant according to any of those described herein, an elongate housing and a cannula extending longitudinally from the housing, the cannula having a proximal end, a distal sharp end, and a lumen extending therethrough, the lumen having an inner diameter sufficient to receive the intraocular implant and permit translation of the implant through the lumen and into the eye of the patient. The cannula may be a 25 gauge, 26 gauge, 27 gauge, 28 gauge, 29 gauge, or 30 gauge needle, or may otherwise be described as having inner and outer diameters equivalent to those of a 25 gauge, 26 gauge, 27 gauge, 28 gauge, 29 gauge, or 30 gauge needle. The needle, in addition, may be a thin-wall or ultra-thin-wall needle.

According to some embodiments, an intraocular implant is administered intracamerally, into the anterior chamber of the eye of a patient in need thereof and designed to provide a controlled and sustained release of bimatoprost (also referred to as “BimSR”), a highly effective IOP lowering agent, to the anterior chamber of the eye for approximately 3 to 4 months for the reduction of TOP. The polymer matrix of BimSR slowly degrades so that there is no need to remove the implant once the drug has been released. Based on in vitro beagle dog data described in US Patent Application Publication No. 2015-0118279, the BimSR implant was expected to have a drug-release duration of approximately 3 to 4 months, associated with TOP lowering during the same duration.

### EXAMPLES

### Example 1

A phase 1/2, prospective, 24-month, paired-eye clinical trial was conducted in 75 glaucoma patients with study eye baseline mean TOP of 25.2 (range 22-36) mmHg after washout. An intraocular implant having the composition as described in US Patent Publication No. 2015-0118279 as formulation 2, as described in TABLE 1 of the specification (6, 10, 15, or 20 μg dose strength) was administered intracamerally in the study eye; the fellow eye was treated with topical bimatoprost solution 0.03% once a day. Long-term TOP-lowering effect was assessed by TOP, time to additional TOP-lowering treatment, and percentage of patients without additional TOP-lowering treatment. Patients were permitted rescue topical medication or a single repeat treatment with BimSR. Results are presented for the 10 and 15 μg dose strengths of BimSR (n=21 for each) that are continuing in development.

Up to 4 and 6 months, 95.2% and 66.7% of patients, respectively, were controlled on the initial BimSR 10 or 15 μg treatment without need for rescue medication or implant retreatment. The median time to additional TOP-lowering treatment after BimSR administration was 38-39 weeks. Surprisingly, at month 24, 10/42, 23.8% of patients were still maintained on the initial implant treatment, and mean (SD) TOP was 16.0 (2.2) mmHg and 15.9 (2.5) mmHg for the BimSR 10 μg and 15 μg dose strengths, respectively, compared with 16.4 (2.1) mmHg and 15.4 (2.4) mmHg for the bimatoprost solution-treated fellow eyes. Adverse events reported were consistent with what would be expected from the administration procedure and with use of topical TOP-lowering medication.

Similar results were observed at dose strengths of 6 μg and 20 μg.

A single administration of BimSR 10 or 15 μg controlled TOP up to 6 months in 66.7% of patients and 23.8% of patients at 24 months. Mean TOP at Month 24 was comparable in eyes maintained on a single administration of BimSR 10 μg or 15 μg and fellow eyes treated with bimatoprost solution. BimSR has demonstrated favorable TOP-lowering efficacy with effects of a single implant lasting for 6 months in the majority of patients and up to 24 months in a subset of patients.

The longevity of effect of a single administration of BimSR is advantageous in addressing nonadherence to daily topical treatment, and in eliminating the need for long term use of TOP lowering medication(s) in patients with glaucoma or ocular hypertension.

These results from the clinical study are unexpected based on the pharmacokinetic profile of the implant in animal studied, supporting an TOP lowering effect of 3-4 months.

Studies in dogs using BimSR have shown a dose related duration of action (for TOP lowering) ranging from 2 months to approximately 4 months with dose strengths of 8-30 μg. Similar duration of effect was seen in monkeys. In another study, animals treated with a single intracameral 20 μg BimSR implant, bimatoprost and its main metabolite, bimatoprost acid, were detected up to Week 10 in aqueous humor at concentrations similar to that seen following topical exposure in humans 2 (Table 3). At 14 weeks postdose, bimatoprost was also detected in cornea, choroid, and iris-ciliary body; however, drug levels in the retina and vitreous humor were below the limit of quantitation. Bimatoprost acid was not detected in ocular tissues. No drug was detected in the remnant implants collected at 14 weeks postdose.

TABLE 3
The Ocular and Systemic Pharmacokinetic Data With Bimatoprost SR in Comparison
With Topical and Other Routes of Administration of Bimatoprost

| | | | Systemic | | Aqueous Humor | | Tissues Concentration | | |
|---|---|---|---|---|---|---|---|---|---|
| | | | Concentration | | Concentration | | (ng/g) | | |
| | | | (ng/mL) | | (ng/mL) | | Cornea | ICB | Choroid |
| Route of | Species | | AGN- | AGN- | AGN- | AGN- | AGN- | AGN- | AGN- |
| Administration | Study Number | Dose | 192024 | 191522 | 192024 | 191522 | 192024 | 192024 | 192024 |
| Intracameral | Dogs | | | | | | | | |
| | TX12102 | 10, 15 & 20 μg | BLQ^{a} | BLQ^{a}- | — | — | — | — | — |
| | | | | 0.0673 | | | | | |
| | PK11086^{b} | 20 μg | — | — | 13.7 | 3.4 | 4.62 | 2.8 | 0.942 |
| | Monkey | | | | | | | | |
| | TX09051 | 30 μg (Gen 1) | 0.280 | 0.165 | — | — | — | — | — |
| | Human^{c} | | | | | | | | |
| | 192024-041D | 6, 10, 15 & 20 μg | BLQ- | BLQ- | — | — | — | — | — |
| | | | 0.00502 | 0.0245 | | | | | |
| Topical | Dog | | | | | | | | |
| | PK10130 | 0.03% QD | — | — | BLQ^{d} | 2.0 | — | — | — |
| | Monkey | 0.03% QD | 0.397 | BLQ^{e} | — | — | — | — | — |
| | 6177-110 | 0.1% BID | 1.92 | BLQ^{e} | — | — | — | — | — |
| | PK-98-003 | 0.1% QD | — | — | 34.5 | — | 558 | 797 | — |
| | PK-97-032 | 0.1% BID | — | — | 13.0 | — | 2890 | 1369 | — |
| | Human | | | | | | | | |
| | PK-98-119 | 0.03% QD | 0.0822 | 2.41 | — | — | — | — | — |
| | Camras, 2004^{f} | 0.03% QD | — | — | 2.37 | 8.55 | — | — | — |

BID = twice daily;
BLQ = below the limit of quantitation;
Gen = Generation;
ICB = ins-cihary body;
QD = once daily;
— = Data not collected
^{a}Lower limit of quantitation (LLOQ) = 0.025 ng/mL (AGN-192024) and 0.05 ng/mL (AGN-191522) in blood
^{b}AGN-192024 or AGN-191522 BLQ in vitreous and retina.
^{c}LLOQ = 0.001 ng/mL (AGN-192024) and 0.010 ng/mL (AGN-191522) in plasma; preliminary data from 299 samples from 57 patients in the study
^{d}LLOQ = 0.200 ng/mL (AGN-192024) in aqueous humor
^{e}LLOQ = 0.100 ng/mL (AGN-191522) in blood
^{f}Camras et al, 2004

### Example 2

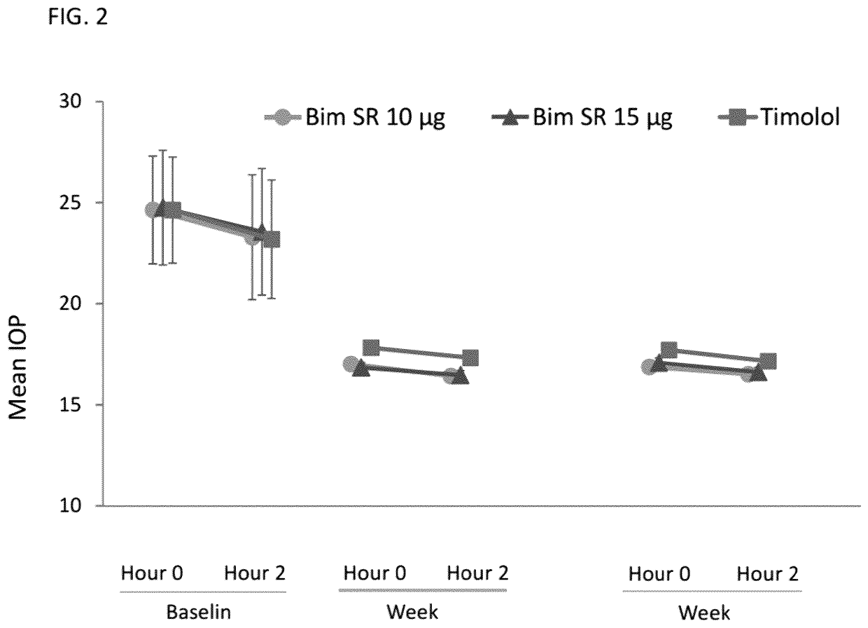

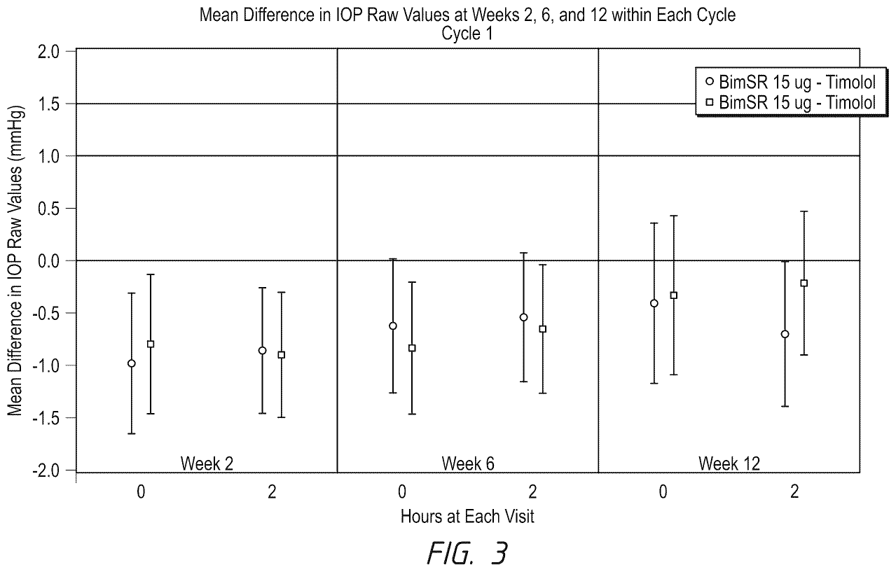

In one example, in a Phase 3 multicenter, randomized, masked, parallel-group comparison of BimSR versus active control (timolol maleate 0.5% eye drops; referred as Timolol) intraocular pressure (IOP)-lowering efficacy and safety of two dose strengths of BimSR in patients with open-angle glaucoma (OAG) or ocular hypertension (OHT) were studied after initial and repeated administrations. Relevant details and results are shown in FIGS. 1-4. The BimSR implant was an intraocular implant having the composition as described in US Patent Publication No. 2015-0118279 as formulation 2, as described in TABLE 1 of the specification (10 μg or 15 μg dose strength). As shown in Table 4 below, the patients were randomized into three groups and the groups either received BimSR 10 μg intracameral implant, BimSR 15 μg intracameral implant, or Timolol drops BID. Patients in the groups receiving one of the two BimSR implant strengths received a single implant via injection into the patients' anterior chamber in three administration cycles at day 1, week 16 (month 4), and week 32 (month 8) during a 12 month treatment period, with safety follow-up to month 20.

TABLE 4

| Treatment | | Fellow Eye |
|---|---|---|
| Group | Study Eye Treatment | Treatment |
| Bim SR 10 μg | Dose strength: 10 μg | Sham administration |
| | Eye drops: | procedure |
| | Vehicle BID | Eye drops: Timolol BID |
| Bim SR 15 μg | Dose strength: 15 μg | Sham administration |
| | Eye drops: | procedure |
| | Vehicle BID | Eye drops: Timolol BID |
| Timolol | Sham administration | Sham administration |
| | procedure | procedure |
| | Eye drops: Timolol BID | Eye drops: Timolol BID |

The two strengths of BimSR reduced TOP by approximately 30 percent over the 12-week primary efficacy period, meeting the predefined criteria for non-inferiority to Timolol. The two strengths of BimSR were also well tolerated in the majority of patients. Specifically, with Bim SR 15 μg, the mean difference (versus timolol) in the study eye TOP ranged from −0.98 to −0.41 mm Hg; the upper limit of the 95% CI is ≤1.0 mm Hg at all 6 timepoints analyzed over the 12 weeks during each treatment cycle. For BimSR 10 μg the mean difference (versus timolol) in the study eye TOP ranged from −0.90 to −0.21 mm Hg; the upper limit of the 95% CI for the mean difference is ≤1.0 mm Hg at all 6 timepoints analyzed over the 12 weeks during each treatment cycle.

After three treatment cycles, patients who were 12 months or more past cessation of treatment were analyzed. For those patients who underwent 3 administration cycles (i.e., who received a total of three implants, one implant at day 1, one implant at week 16, and one implant at week 32), 90% of patients treated with Bim SR 15 μg dose, and 83% of patients treated with Bim SR 10 μg dose, were not on rescue treatment for at least 360 days (12 months) after the third administration. Rescue medications refers to the need for non-study intraocular pressure lowering medication to control elevated TOP. Bim SR has demonstrated favorable IOP-lowering efficacy with effects of three implants, each administered once every 4 months, lasting for at least 12 months after the third injection in a majority of patients studied.

Similarly, after the last administration of Bim SR (1 or 2 or 3 administration cycles), 76% of patients treated with Bim SR 15 μg dose and 76% of patients treated with Bim SR 10 μg dose, were not on rescue treatment for at least 360 days (12 months). Bim SR has demonstrated favorable IOP-lowering efficacy with effects of one, two, or three implants lasting for at least 12 months after the final injection in a majority of patients who were studied.

1. A method of reducing intraocular pressure (TOP) in the eye of a patient comprising:
injecting a single intraocular implant comprising bimatoprost or a salt thereof and a biodegradable polymer into the anterior chamber of a patient in need thereof;
wherein the intraocular implant is effective to reduce the IOP of the patient in need thereof over a period of time between about 12 months and about 24 months.

2. The method of claim 1, wherein the intraocular implant is effective to reduce the IOP of the patient in need thereof of a period of time of about 24 months.

3. The method of claim 1, wherein the intraocular implant comprises 6 μg, 10 μg, 15 μg or 20 μg of bimatoprost or a salt thereof.

4. The method of claim 3, wherein the intraocular implant comprises a biodegradable polymer matrix, polyethylene glycol 3350, and bimatoprost or a salt thereof, wherein the bimatoprost or a salt thereof and polyethylene glycol 3350 are associated with the biodegradable polymer matrix, which comprises
a) an ester end poly(D,L-lactide) having an inherent viscosity of 0.25-0.35 dl/g,
b) an acid end poly(D,L-lactide) having an inherent viscosity of 0.16-0.24 dl/g, and
c) an ester end poly(D,L-lactide-co-glycolide) having an inherent viscosity of 0.16-0.24 dl/g and a D,L-lactide to glycolide molar ratio of about 75:25;
wherein the bimatoprost or a salt thereof constitutes 18 to 22% of the implant by weight, the ester end poly(D,L-lactide) constitutes 18 to 22% of the implant by weight, the acid end poly(D,L-lactide) constitutes 13.5 to 16.5% of the implant by weight, the ester end poly(D,L-lactide-co-glycolide) constitutes 36 to 44% of the implant by weight, and wherein the polyethylene glycol 3350 constitutes 3.5 to 6.5% of the implant by weight, wherein the inherent viscosity of each of the poly(D,L-lactide) and poly(D,L-lactide-co-glycolide) polymers is determined for a 0.1% solution of the polymer in chloroform at 25° C.

5. A method of treating open angle glaucoma or ocular hypertension in a patient comprising:
injecting one or more single intraocular implants comprising bimatoprost or a salt thereof and a biodegradable polymer into the anterior chamber of the eye of a patient in need thereof at a frequency of one intraocular implant every four months to one intraocular implant every twelve months over a treatment period comprising injection of a first intraocular implant and a final intraocular implant;
wherein the intraocular implants are effective to reduce the TOP of the patient in need thereof over a period of time between about 12 months and about 24 months after injection of the final intraocular implant.

6. The method of claim 5, wherein the patient receives one intraocular implant every four months; and wherein the intraocular implants are effective to reduce the TOP of the patient in need thereof over a period of time between about 12 months and about 24 months.

7. The method of claim 6 wherein the patient receives between two implants and eight implants over the treatment period.

8. The method of claim 6 wherein the patient receives three implants.

9. The method of claim 8 wherein the intraocular implants are effective to reduce the TOP of the patient in need thereof over a period of time of about 12 months.

10. The method of claim 9, wherein the intraocular implant comprises 6 μg, 10 μg, 15 μg, or 20 μg of bimatoprost or a salt thereof.

11. The method of claim 10, wherein the intraocular implant comprises a biodegradable polymer matrix, polyethylene glycol 3350, and bimatoprost or a salt thereof, wherein the bimatoprost or a salt thereof and polyethylene glycol 3350 are associated with the biodegradable polymer matrix, which comprises
(a) an ester end poly(D,L-lactide) having an inherent viscosity of 0.25-0.35 dl/g,
(b) an acid end poly(D,L-lactide) having an inherent viscosity of 0.16-0.24 dl/g, and
(c) an ester end poly(D,L-lactide-co-glycolide) having an inherent viscosity of 0.16-0.24 dl/g and a D,L-lactide to glycolide molar ratio of about 75:25;
wherein the bimatoprost or a salt thereof constitutes 18 to 22% of the implant by weight, the ester end poly(D,L-lactide) constitutes 18 to 22% of the implant by weight, the acid end poly(D,L-lactide) constitutes 13.5 to 16.5% of the implant by weight, the ester end poly(D,L-lactide-co-glycolide) constitutes 36 to 44% of the implant by weight, and wherein the polyethylene glycol 3350 constitutes 3.5 to 6.5% of the implant by weight, wherein the inherent viscosity of each of the poly(D,L-lactide) and poly(D,L-lactide-co-glycolide) polymers is determined for a 0.1% solution of the polymer in chloroform at 25° C.

12. The method of claim 11, wherein the method is effective to reduce the TOP of the patient in need thereof by about 30% from baseline.

13. A method of treating open angle glaucoma or ocular hypertension in a patient comprising:
injecting one or more single intraocular implants comprising bimatoprost or a salt thereof and a biodegradable polymer into the anterior chamber of the eye of a patient in need thereof at a frequency of one intraocular implant every four months to one intraocular implant every twelve months over a treatment period comprising injection of a first intraocular implant and a final intraocular implant;
wherein, after injection of the final implant, the patient does not require rescue medication for a period of time between about 12 months and about 24 months after injection of the final intraocular implant.

14. The method of claim 13, wherein the patient receives one intraocular implant every four months; and wherein the patient does not require rescue medication over a period of time between about 12 months and about 24 months after injection of the final implant.

15. The method of claim 14 wherein the patient receives between two implants and eight implants over the treatment period.

16. The method of claim 15 wherein the patient receives three implants.

17. The method of claim 16 wherein the patient does not require rescue medication over a period of time of about 12 months.

18. The method of claim 17, wherein the intraocular implant comprises 6 μg, 10 μg, 15 μg, or 20 μg of bimatoprost or a salt thereof.

19. The method of claim 18, wherein the intraocular implant comprises a biodegradable polymer matrix, polyethylene glycol 3350, and bimatoprost or a salt thereof, wherein the bimatoprost or a salt thereof and polyethylene glycol 3350 are associated with the biodegradable polymer matrix, which comprises
(a) an ester end poly(D,L-lactide) having an inherent viscosity of 0.25-0.35 dl/g,
(b) an acid end poly(D,L-lactide) having an inherent viscosity of 0.16-0.24 dl/g, and
(c) an ester end poly(D,L-lactide-co-glycolide) having an inherent viscosity of 0.16-0.24 dl/g and a D,L-lactide to glycolide molar ratio of about 75:25;
wherein the bimatoprost or a salt thereof constitutes 18 to 22% of the implant by weight, the ester end poly(D,L-lactide) constitutes 18 to 22% of the implant by weight, the acid end poly(D,L-lactide) constitutes 13.5 to 16.5% of the implant by weight, the ester end poly(D,L-lactide-co-glycolide) constitutes 36 to 44% of the implant by weight, and wherein the polyethylene glycol 3350 constitutes 3.5 to 6.5% of the implant by weight, wherein the inherent viscosity of each of the poly(D,L-lactide) and poly(D,L-lactide-co-glycolide) polymers is determined for a 0.1% solution of the polymer in chloroform at 25° C.

20. The method of claim 19, wherein the rescue medication comprises eye drops containing a prostaglandin analog or prostamide.

21. (canceled)
